(19) United States
(12) Patent Application Publication
(10) Pub. No.: US 2006/0241423 A1
(43) Pub. Date: Oct. 26, 2006

(54) Apparatus and method for full-field breast ultrasound scanning

(76) Inventors: Tor C Anderson, Mountain View, CA (US); Reino E Hautala, Campana Drive, CA (US); Janet B Mar, San Francisco, CA (US)

Correspondence Address: COOPER & DUNHAM, LLP, 1185 AVENUE OF THE AMERICAS, NEW YORK, NY, 10036, US

(21) Appl. No.: 10/528,922

(22) PCT Filed: Oct. 1, 2003
(86) PCT No.: PCT/US03/31434

§ 371 Date: Feb. 8, 2006

Related U.S. Application Data
(60) Provisional application No. 60/415,385, filed on Oct. 1, 2002.
(60) Provisional application No. 60/429,728, filed on Nov. 27, 2002.
(60) Provisional application No. 60/439,437, filed on Jan. 9, 2003.
(60) Provisional application No. 60/480,095, filed on Jun. 20, 2003.

(30) Foreign Application Priority Data
Nov. 27, 2002 (US) 10305661
Nov. 27, 2002 (US) 10305936

(51) Int. Cl. A61B 8/00

(52) U.S. Cl. 600/437

(57) ABSTRACT

A full-field breast ultrasound (FFBU) scanning apparatus and related methods are described for compressing and ultrasonically scanning a breast. A first surface of an at least partially conformable, substantially taut membrane or film sheet compresses one side of the breast, and the other side of the breast is compressed by a compression assembly comprising a rigid compression plate and an inflatable air bladder. A transducer translation mechanism holds a transducer surface against a second surface of the film sheet while translating the transducer thereacross to scan the breast. An irrigation system automatically maintains a continuous supply of coupling agent at an interface between the transducer surface and the film sheet as the transducer is translated. A recycling system collects used coupling agent for re-use by the irrigation system. The transducer is housed in a substantially closed environment to prevent evaporative acoustic couplant loss and to allow scanning at many different angles without couplant loss. A variety of other usability, patient comfort, and safety features are also described.

### CROSS-REFERENCE TO RELATED APPLICATIONS

This application is a continuation-in-part of U.S. Ser. No. 10/160,836 filed May 31, 2002, and claims the benefit of U.S. Provisional Application No. 60/415,385, filed Oct. 1, 2002, each of which is incorporated by reference herein. This patent specification also relates at least in part to subject matter disclosed in the following applications: International Application Ser. No. PCT/US3/13712 filed May 30, 2003; U.S. Ser. No. 60/480,095 filed Jun. 20, 2003; U.S. Ser. No. 60/439,437 filed Jan. 9, 2003; U.S. Ser. No. 60/429,728 filed Nov. 27, 2002; U.S. Ser. No. 10/305,661 filed Nov. 27, 2002; U.S. Ser. No. 10/305,936 filed Nov. 27, 2002; International Application Ser. No. PCT/US01/43237, filed Nov. 19, 2001; U.S. Ser. No. 60/326,715 filed Oct. 3, 2001; and U.S. Ser. No. 60/252,946 filed Nov. 24, 2000, each of which is incorporated by reference herein.

### FIELD

This patent specification relates to ultrasonic imaging of the breast. More particularly, this patent specification relates to an apparatus and related methods for acquiring ultrasound scans of a compressed breast for use in adjunctive ultrasound mammography or other applications requiring reliable and repeatable three-dimensional breast ultrasound data.

### BACKGROUND

X-ray mammography is currently the only imaging method used in en masse breast cancer screening environments. In health maintenance organizations (HMOs) and other medical organizations, specialized x-ray mammography clinics designed for high patient throughput are being increasingly used to screen as many women as possible in a time and cost efficient manner. Numerous studies have shown that early detection saves lives and increases treatment options. Recent declines in breast cancer mortality rates (e.g., 39,600 deaths in 2002 versus 41,200 in 2000) have been attributed, in large part, to the regular use of screening x-ray mammography.

It has been found that the use of ultrasound mammography (sonomammography) in conjunction with conventional x-ray mammography can drastically increase the early breast cancer detection rate. Whereas x-ray mammograms only detect a summation of the x-ray opacity of individual slices over the entire breast, ultrasound can separately detect the acoustic impedance of individual slices of breast tissue, and therefore may allow detection of breast lesions where x-ray mammography alone fails.

Devices for facilitating breast ultrasound scans have been proposed in which the breast is held still between the inner surfaces of upper and lower compressive members while an ultrasound transducer is swept across an outer surface of one of the compressive members. Because the breast is held motionless during the movement of the ultrasound probe, a three-dimensional volumetric representation of the breast may be constructed from the acquired readings.

Examples of proposed devices for breast ultrasound scanning are discussed in: U.S. Pat. No. 5,660,185 and U.S. Pat. No. 5,664,573, which discuss ultrasound-assisted biopsy procedures; U.S. Pat. No. 6,027,457, which discusses a combined x-ray mammography and ultrasound mammography apparatus; WO 83/02053, which discusses an apparatus for ultrasonic examination of deformable objects such as the female human breast, and U.S. Pat. No. 6,574,499, which discusses an apparatus for generating breast ultrasound image data in spatial registration with x-ray mammography data.

In order for a breast ultrasound scanning unit to be highly effective in an en masse breast cancer screening environment, several important issues relating to image quality, repeatability, system cost, spatial practicality, and workflow-related practicality should be addressed. It is believed that each of the above proposals fails to address at least one of these issues, and other issues identified herein, that cause it to be less useful in an en masse breast cancer screening environment than the systems and methods described herein. It is to be appreciated, however, that the systems and methods of the present disclosure may be suitable for a variety of other medical imaging applications other than en masse breast cancer screening.

As described in parent application U.S. Ser. No. 10/160,836, supra, it is desirable to compress the breast along a standard x-ray mammogram view plane such as the craniocaudal (CC) or mediolateral oblique (MLO) view. Such placement and compression of the breast promotes repeatability and also provides for ready comparison with x-ray mammogram views of the breast. Compression of the breast also reduces the required ultrasonic penetration, therefore yielding better image quality. However, at the same time, it is necessary to maintain as much acoustic coupling as possible between the ultrasound probe and the compressed breast. Even very small air gaps in the acoustic path between the ultrasound transducer and the breast tissue can cause unacceptable amounts of attenuation. More generally, any kind of acoustic impedance mismatch along the acoustic path between the piezoelectric transducer elements and the target tissue can reduce image quality.

The above design challenges are made even more challenging by the many practical issues in real-world clinical screening environments. The ultrasound scanning process should be technician-friendly and should reduce the probability and/or severity of human errors with respect to both image quality and patient comfort. The overall breast ultrasound scanning process, including patient preparation, breast positioning, breast scanning, and inter-patient equipment recovery and maintenance should be as time-efficient as possible. Other relevant issues include footprint requirements (the smaller the better), general appearance, acquisition costs, maintenance costs, and the amount and nature of consumables used per patient.

Accordingly, it would be desirable to provide a full-field breast ultrasound (FFBU) scanning apparatus and related methods that obtain high-quality volumetric ultrasounds of a breast for use in adjunctive ultrasound mammography, computer-aided diagnosis, or other medical applications.

It would be further desirable to provide an FFBU scanning unit that compresses the breast with reduced patient discomfort while also facilitating thorough ultrasonic scanning thereof including areas near the breast periphery.

It would be still further desirable to provide an FFBU scanning unit that effectively compresses the breast while also minimizing acoustic attenuation losses, reverberation artifacts, and other image quality degradations that can be caused by interference in the acoustic path between an ultrasound transducer and the target breast tissue.

It would be even further desirable to provide an FFBU scanning unit that is safe and easy to use, that is comfortable to the patient, that is robust against human error and/or reduces the likelihood of human error, and that provides standardized and repeatable ultrasonic breast scans.

### SUMMARY

A full-field breast ultrasound (FFBU) scanning apparatus and related methods are provided for compressing a breast and ultrasonically scanning the compressed breast volume. The FFBU scanning apparatus comprises an at least partially conformable membrane or film sheet in a substantially taut state, and further comprises a compression assembly movable relative to the film sheet to allow placement and compression of a breast therebetween, the breast being compressed against a first surface of the film sheet. The FFBU scanning apparatus further comprises a transducer translation mechanism configured to hold a surface of an ultrasound transducer against a second surface of the film sheet while translating the ultrasound transducer thereacross to scan the breast, and an irrigation system for automatically maintaining a continuous supply of coupling agent at an interface between the transducer surface and the film sheet as the ultrasound transducer is translated across the film sheet.

Preferably, the coupling agent comprises a substantially nonviscous liquid such as water. A frame sealably encloses the ultrasound transducer in cooperation with the film sheet for preventing loss of the nonviscous liquid coupling agent. A coupling agent recycling system is provided that collects coupling agent that falls away or otherwise departs the interface between the film sheet and the transducer surface, and returns the coupling agent to the irrigation system for reapplication to that interface. A wicking or capillarity-based effect draws the coupling agent between the scanning surface and the film sheet for minimizing attenuation losses or artifacts due to tiny air pockets that would otherwise exist at the interface between the film sheet and the transducer surface. The film sheet and the scanning surface should be acoustically matched.

The frame housing and compression assembly are rotatable around an anterior-posterior axis of a patient for facilitating breast scans at different scan angles including a CC angle, an MLO angle, and an ML angle. The coupling agent recycling system is configured to collect and return coupling agent to the irrigation system regardless of the particular angle of the scan. Preferably, the ultrasound transducer is a linear array transducer having a sufficient length (e.g., 15 cm) to allow the breast to be completely imaged in a single imaging sweep.

The compression assembly comprises a substantially rigid plate that applies most of a total compression weight to the breast. The compression assembly further comprises an inflatable bladder that applies a remainder of the total compression weight to the breast in a peripheral area near a skinline of the compressed breast, thereby increasing the amount of breast that can be scanned near the skinline.

A method for scanning a breast is also provided that facilitates patient comfort by reducing scanning time without sacrificing image quality. Prior to a full-resolution imaging sweep of the ultrasound transducer across the breast, for which full-resolution frames are captured at closely spaced transducer locations corresponding to a desired image resolution, a relatively brief survey sweep is performed having reduced-resolution frames and coarser spacing between transducer locations. Information acquired during the survey sweep is processed to establish the lateral extent of the breast volume in the lateral direction, i.e., in the direction of transducer movement, as well as the axial extent of the breast away from the patient's body, i.e. in a direction along the transducer axis. A full-resolution imaging sweep is then performed, during which lateral areas on either side of the breast volume are that were identified during the survey sweep are skipped to reduce scanning time, and during which piezoelectric elements on the transducer lying axially outside of the breast volume are not fired, thereby further saving scanning time. Preferably, the survey images are also used to establish, in an AGC (automatic gain control) process, optimal transmit and receive parameters that can obtain the best signal-to-noise ratio (SNR) for each image pixel and image uniformity among the pixels.

According to another preferred embodiment, the thickness of the compressed breast, i.e., the distance between the compression plate and the film sheet is automatically sensed using mechanical sensors. Knowledge of the breast thickness is used to further reduce scanning time by obviating the need to image beyond that known distance. A variety of other comfort, usability, and safety features are provided as described herein.

### BRIEF DESCRIPTION OF THE DRAWINGS

FIG. 1 illustrates a perspective view of a full-field breast ultrasound (FFBU) scanning unit according to a preferred embodiment;

FIG. 2 illustrates a perspective view of a breast compression and scanning assembly corresponding to the FFBU scanning unit of FIG. 1;

FIG. 3 illustrates a conceptual side cutaway view of the breast compression and scanning assembly of FIG. 2 as it scans a compressed breast;

FIGS. 4A and 4B illustrate perspective views of a frame of an ultrasound scanning assembly corresponding to the breast compression and scanning assembly of FIG. 2 with an ultrasound probe assembly removed and inserted, respectively;

FIG. 5 illustrates a perspective view of a probe assembly according to a preferred embodiment;

FIG. 6 illustrates an axial cutaway view of the probe assembly of FIG. 5;

FIG. 7 illustrates a conceptual cutaway axial view of the probe assembly of FIG. 6 as it performs an ultrasound scan of a breast; and

FIG. 8 illustrates step for performing a full-field ultrasound scan of a breast according to a preferred embodiment.

### DETAILED DESCRIPTION

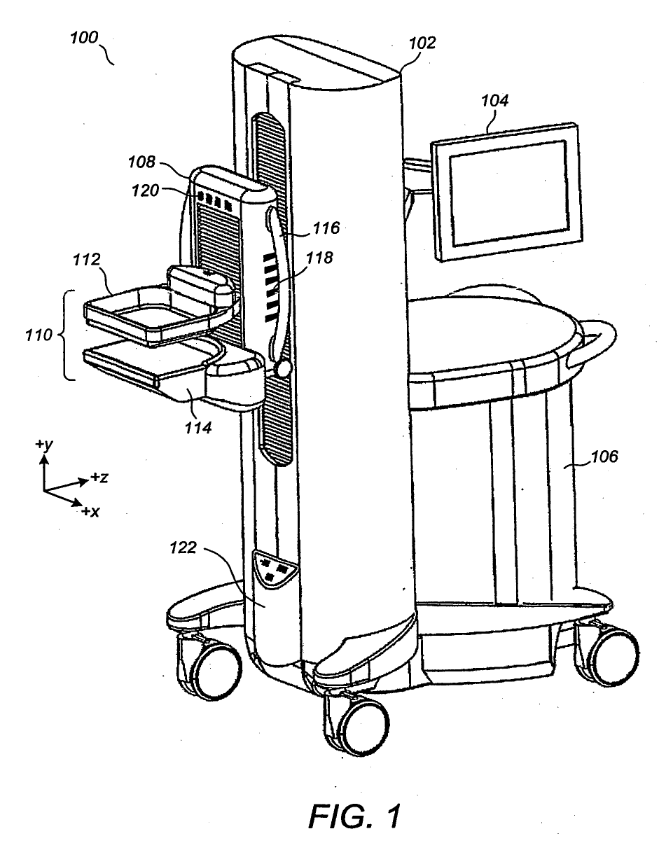

FIG. 1 illustrates a perspective view of a full-field breast ultrasound (FFBU) scanning unit 100 according to a preferred embodiment. FFBU scanning unit 100 comprises a housing 102 that, from a visual and mechanical perspective, is reminiscent of the “look and feel” of many conventional x-ray mammography units being marketed today. In addition to addressing functional and practical concerns such as machine footprint size, the familiar appearance may promote faster clinician acceptance of FFBU scanning as a standardized adjunct to x-ray mammography.

A display monitor 104 provides for user input and real-time feedback during the scanning process. The display monitor 104 may be a touch-screen monitor and/or a keyboard/mouse (not shown) may be provided. Near location 106, FFBU scanning unit 100 comprises a fully functional ultrasound engine for driving an ultrasound transducer and generating volumetric breast ultrasound data therefrom. The volumetric scan data can be transferred to another computer system for further processing using any of a variety of data transfer methods known in the art. A general purpose computer, which can be implemented on the same computer as the ultrasound engine, is provided for general user interfacing and system control. The general purpose computer can be a self-contained stand-alone unit, or can be remotely controlled, configured, and/or monitored by a remote station connected across a network.

FFBU scanning unit 100 movably supports a gantry 108 that in turn supports a breast compression and scanning assembly 110. The gantry 108 is vertically movable for accommodating patients of different heights, including patients in wheelchairs. Breast compression and scanning assembly 110 comprises a compression assembly 112 and a scanning assembly 114, the compression assembly 112 being positioned above (i.e., in the +y direction of FIG. 1) the scanning assembly 114 according to a preferred embodiment.

It has been found that providing the scanning assembly 114 beneath the breast and scanning upward is preferable to providing the scanning assembly 114 above the breast and scanning downward, insofar as gravity urges the breast downward for better acoustic contact across a larger area. However, depending on the size of the breast and other factors, in other preferred embodiments the breast is scanned in a downward direction from above. Advantageously, the gantry 108 is rotatable from −180 degrees to +180 degrees around the z-axis in FIG. 1, i.e. around an axis parallel to an anterior-posterior direction. This allows scanning from any angle. The gantry 108 can be rotated, automatically and/or manually, to any angle for allowing, for example, mediolateral oblique (MLO) scans of either breast, including purely medial-lateral (ML) scans at −90 degrees and +90 degrees. According to the preferred embodiments as described further infra, the breast compression and scanning assembly 110 obtains high-quality scans of the breast for any angle between −180 degrees and 180 degrees, inclusive.

Gantry 108 further comprises handles 116 and position control buttons 118 similar to those provided on commercial x-ray mammography units. In addition, front-mounted scan control buttons 120 are provided on the front of the gantry 108 that can be easily reached by the operator while standing immediately next to the patient. In contrast to x-ray mammography scenarios in which the operator needs to step away from the patient toward the back side of the unit to avoid radiation exposure, ultrasound scanning involves no harmful radiation. According to a preferred embodiment, the front-mounted scan control buttons 120 advantageously allow the operator to control substantially the entire scanning process (starting, stopping, restarting, monitoring, etc.) without leaving the patient's side. Foot pedals (not shown) may also be provided for facilitating control of the breast placement, compression and/or scanning process. According to another preferred embodiment, user input is made easier for MLO or ML views by making the angle of the gantry 108 automatically detected, wherein knowledge of the angle automatically determines which breast is being scanned so that the user is not required to input this information.

Provided near a bottom location 122 of the housing 102 is drawer-like access to a coupling agent recycling station (not shown). As described further infra, an acoustic coupling agent such as water is recyclably applied to an interface between an ultrasound probe and a one side of a taut film sheet, the other side of the taut film sheet compressing the breast. Provided in the coupling agent recycling station is a reservoir and a plurality of pumps, filters, and the like as required to reliably provide the liquid flow and recycling functionalities described infra. The liquid coupling agent recycling station is coupled to the scanning assembly 114 via appropriate plumbing materials and pathways (e.g., Tygon tubing), that could be readily realized by one skilled in the art in view of the present disclosure. In view of the very low flow rate required using a preferred interface-wetting system described infra, e.g., 20 ml-150 ml per minute or less, only a modest amount of liquid (e.g., 1 liter) needs to be maintained in the coupling agent recycling system, which is preferably a self-contained, closed system requiring little maintenance.

Although the use of any of a variety of liquid coupling agents is within the scope of the preferred embodiments, better results are obtained when a highly non-viscous liquid is used, such as water. However, it is to be appreciated that other non-viscous, acoustically conductive, well-matched liquids such as glycol could be substituted, provided that their characteristics are analogous to water in terms of their ability to be transported, emitted, pumped, stored, and naturally drawn by wicking, capillarity, and/or surface tension into small spaces. Preferably, the water is treated with an antibacterial agent such as chlorhexadine gluconate, for sanitation purposes, as well as an antifoaming agent to reduce bubbles in the water. In one preferred embodiment, the water is heated to body temperature for increased patient comfort during scanning.

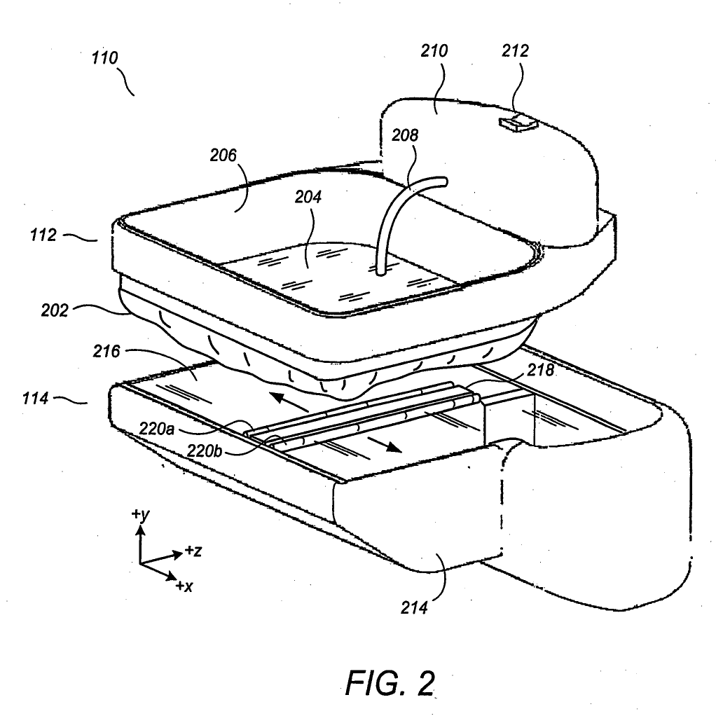

FIG. 2 illustrates a perspective view of the breast compression and scanning assembly 110 including the compression assembly 112 and the scanning assembly 114. Compression assembly 112 comprises a frame 206 housing a compression plate 204 and having a bladder 202 formed by sealing a loose silicone rubber sheet around a bottom periphery of the compression plate 204. The silicone rubber sheet can be sealed to the compression plate using silicone RTV adhesive/sealant. In operation, the bladder 202 is filled with air to compress the periphery of the breast against an upper surface of the scanning assembly 114. In one preferred embodiment, the silicone rubber sheet is approximately 0.01 inches thick.

It is to be appreciated that although the terms “upper,” “lower,” “top,” and “bottom” are used to describe the various components of the breast compression and scanning assembly 110, these terms are not to be construed as limiting the orientation thereof. As described supra, the breast compression and scanning assembly 110 can be placed at any angle between −180 and 180 degrees around the z-axis of FIGS. 1 and 2 and can compress and scan the breast at any of those angles.

Compression assembly 112 further comprises an air pressure/vacuum supply housing 210 that houses an air pump (not shown) and solenoid valve (not shown) coupled to the bladder 202 by an air tube 208. The air pressure/vacuum supply can be manually controlled using a switch 212, and can also be automatically controlled by a control computer. A safety relief valve (not shown) is also provided such that bladder pressures above a predetermined safety limit, such as 1 psi, are avoided. Also, the total downward force on the breast is sensed and monitored, and the air pump is shut off if a predetermined overall load limit is exceeded. An inflation of the bladder to between 0.25-1.0 psi is typically sufficient to achieve good contact of the breast periphery with the surface of the scanning assembly 114.

Preferably, the compression plate 204 is substantially rigid, and both the compression plate 204 and the bladder 202 are translucent so that the patient and the operator can see the upper surface of the breast. Preferably, there are visible markings (not shown) provided on the compression plate 204, such as a center line, to properly guide the placement of the breast onto the top of the scanning assembly 114. The markings may also include sample outlines of compressed breasts at different sizes, so as to guide the breast placement. The markings are also preferably duplicated on the upper surface of the scanning assembly 114.

Although air is used for inflating the bladder 202, other fluids such as oils or non-viscous liquids may be used. In an alternative preferred embodiment, a fluid is used in the bladder 202 that has high acoustic attenuation characteristics and/or is also acoustically well-matched to the breast tissue, whereby reflections from the upper tissue-(silicone)-fluid interface are minimized for increasing image quality even further. In another preferred embodiment, a pressurized reservoir or accumulator maintains a fixed pressure in the bladder 202 at all times.

Scanning assembly 114 comprises a frame 214 having a taut film sheet 216 extending thereover, the frame 214 and film sheet 216 together forming a closed chamber that houses a probe assembly 218. The film sheet 216 is preferably a flexible but non-stretchable material that is thin, water-resistant, durable, highly acoustically transparent, chemically resistant, and biocompatible. In one preferred embodiment, the film sheet 216 comprises a sheet of Melinex® or Mylar® that is 2 mils thick. In another preferred embodiment, the film sheet 216 comprises another type of biaxially oriented polyester film, or another type of material having properties similar to Melinex® or Mylar®. The film sheet 216 is attached to the frame 214 in a substantially airtight manner so as to form a closed environment, thereby inhibiting evaporation of the coupling agent or other forms of coupling agent loss. In one preferred embodiment, the frame 214 comprises a polyethylene terephthalate (PET) lip, and the film sheet 216 is attached to the PET lip using a cyanoacrylate adhesive.

Probe assembly 218 is mechanically coupled to the frame such that it can sweep laterally across the breast (i.e., in the +x/−x direction in FIG. 2) under motor control while its transducer surface is in contact with the film sheet 216. Preferably, the transducer of the probe assembly 218 is a linear array transducer that is sufficiently long, e.g., 15 cm, to obtain a volumetric B-mode scan of the breast in a single sweep.

In one preferred embodiment, the linear array transducer is 146 mm long and comprises 768 piezoelectric elements. The linear array transducer has an operating frequency of 7.5 MHz, although other frequencies ranging from 6 MHz to 10 MHz produce good results, and still other frequencies from 2 MHz to 15 MHz are within the scope of the preferred embodiments. Mechanical focusing is preferred over the use of RTV acoustic lenses, with mechanical focusing yielding comparatively less near-field lens reverberation artifact and reduced attenuation losses. In one preferred embodiment, there are 384 vectors per frame, 192 transmit and receive channels, and multi-zone focusing with 3-4 zones. Typical parameters may include a frame rate of 5-15 frames per second (fps), with a nominal frame rate of 10 fps.

For a full imaging sweep (in distinction to a survey sweep described herein), 600 image slices separated by 0.4 mm may be obtained for a 24 cm-wide volume in a 60-second sweep. According to a preferred embodiment, after the breast is properly positioned and compressed, a brief (e.g., 10-second) survey sweep is performed prior to the imaging sweep. The survey sweep moves the probe assembly at a relatively high speed across the breast, and only a few frames or less per cm are captured. Survey images taken from the survey sweep are then used to establish the lateral extent of the breast in the +x/−x direction and the axial extent of the breast (i.e., in the +z direction) from the chest wall. The survey images are also used to establish, in an AGC (automatic gain control) process, optimal transmit and receive parameters that can obtain the best signal-to-noise ratio (SNR) for each image pixel and image uniformity among the pixels.

According to a preferred embodiment, during the imaging sweep, the ultrasound probe skips over empty lateral areas on either side of the breast that were identified during the survey sweep, thereby decreasing the amount of scan time. Also, piezoelectric elements that correspond axially (i.e., in the +z direction) to empty areas outside the breast are not fired, thereby further decreasing scan time. For example, if the breast has a lateral extent of 16 cm and a depth of 7.5 cm, the above 60-second imaging sweep can be reduced to roughly about (16/24)*(7.5/15)*60=20 seconds. Thus, in this example, total scan time is reduced from 70 seconds (10-second survey sweep plus 60-second imaging sweep) to 30 seconds (10-second survey sweep plus 20-second imaging sweep).

According to another preferred embodiment, the breast compression and scanning assembly 110 is configured to mechanically detect the thickness of the compressed breast, i.e. the distance between the compression plate 204 and the taut film sheet 216. Knowledge of the breast thickness “T” can further save time by obviating the need to image beyond the depth “T.”

According to a preferred embodiment, an irrigation system is provided for automatically maintaining a continuous supply of coupling agent at an interface between the transducer surface and the film sheet 216 as the ultrasound transducer is translated across the film sheet. Probe assembly 218 includes coupling agent distribution tubes 220a and 220b placed immediately adjacent to the transducer surface. Small holes in the distribution tubes 220a and 220b provide a small flow of coupling agent. The distribution tubes 220a and 220b are positioned next to the transducer surface such that small reservoirs of coupling agent are maintained on either side of the transducer surface at all times during the scanning process. The distribution tubes 220a/220b, the transducer surface, and the film sheet 216 are positioned and configured to foster a wicking or capillary effect that keeps the tiny air pockets that might otherwise exist at the film sheet-transducer surface interface filled with coupling agent. In this manner, acoustic coupling between the transducer surface and the target is facilitated and high image quality obtained.

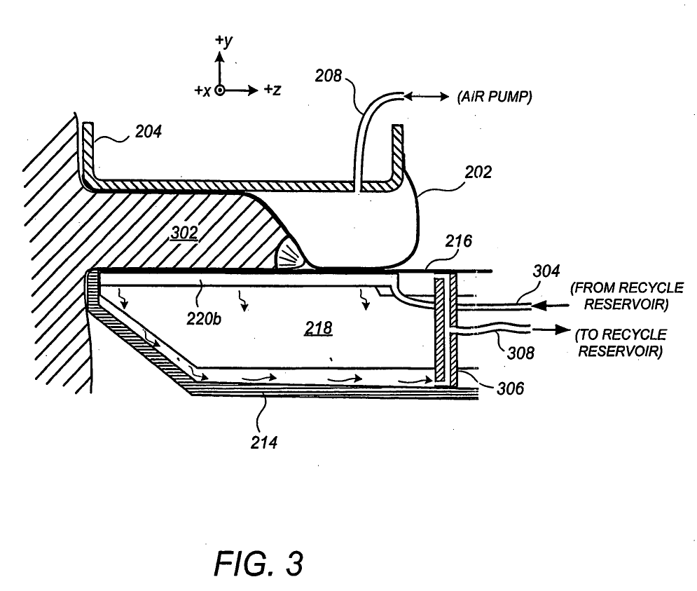

FIG. 3 illustrates a conceptual side cutaway view of a compressed breast 302 as it is being scanned by an FFBU scanning apparatus according to a preferred embodiment. The cutaway sections are at different planes as needed for describing the device. Although depending on the size and characteristics of the breast itself, most of the upper surface of the breast is compressed by the upper compression plate 204. Preferably, the bladder 202 is inflated only after the compression plate has been fully lowered to the final scanning level, i.e., the level at which scanning will take place. This final scanning level usually is achieved when approximately 10-15 total pounds of force has been applied. The bladder 202 serves primarily to urge the periphery of the breast toward the taut film sheet. Generally speaking, this breast periphery would otherwise be suspended in space and therefore not properly imaged by the ultrasound transducer.

Also illustrated in FIG. 3 is a conceptual diagram of the closed-system chamber that is formed by the frame 214 and the film sheet 216. Coupling agent from the recycling reservoir is pumped via a source tube 304 into the distribution tube 220b, which may be made of brass. It is important that coupling agent is not emitted from the distribution tube 220b too fast, or else the film sheet 216 will to “inflate” or rise up above the transducer surface by one millimeter or more by virtue of the fluid pressure, which may reduce image quality. Even if air bubbles are not present, the image quality can be reduced as reverberation artifacts are incurred due to the undesired gap between the probe surface and the taut film sheet. In one preferred embodiment in which 3 1-mm holes are drilled along the length of the distribution tube 220b, and in which the distribution tube 220b has an inner diameter of 5 mm and an outer diameter of 5.32 mm, a water pressure of about 10 psi is suitable.

Coupling agent slowly leaks away from the small “weeping” reservoir maintained near the probe-film sheet intersection, and falls to the bottom of the frame 214. The bottom of the frame 214 is angled slightly so as to urge the coupling agent to flow toward a vertically symmetric drain element 306. The drain element 306 is connected to a vacuum source in the coupling agent recycling system so that the coupling agent is suctionably returned to the recycling reservoir. The drain element 306 is vertically symmetric so that the coupling agent is properly recycled even where the entire assembly of FIG. 3 is turned upside down.

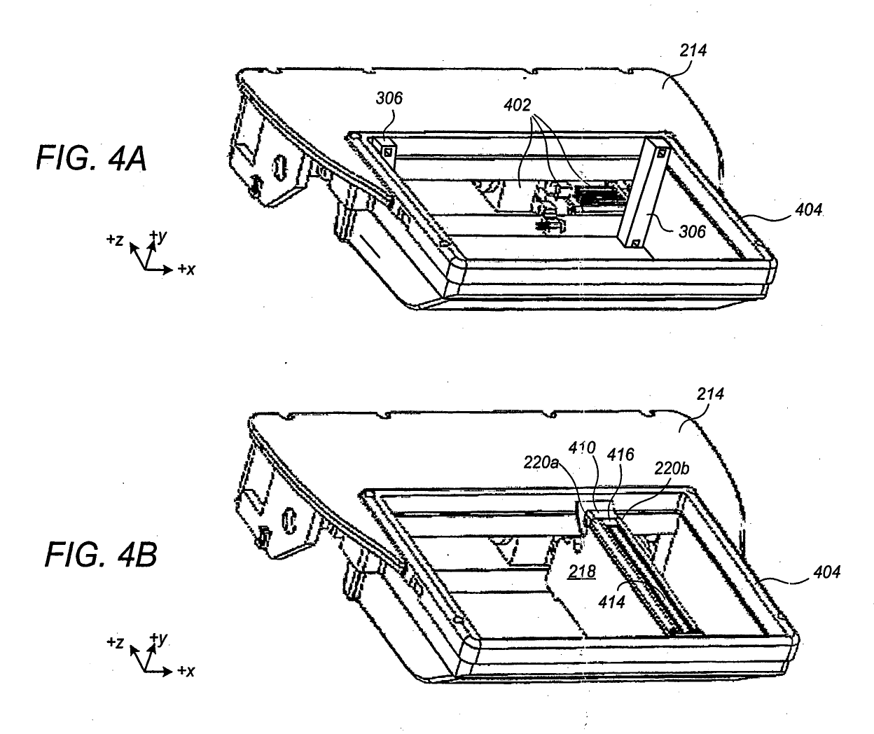

FIGS. 4A and 4B illustrate perspective views the frame 214 with the probe assembly 218 removed and inserted, respectively. As illustrated in FIG. 4A, there are two (2) drain elements 306 provided on each side of the frame 214, thereby providing effective coupling agent return and recycling regardless of the angle of the scanning assembly around the z-axis. Also visible in FIG. 4A is part of a translation mechanism 402 used to translate the probe assembly 218, and a PET plastic lip 404 across which the taut film sheet is placed.

FIG. 4B omits the drain elements 306 and includes the probe assembly 218. Visible in FIG. 4B is a distribution tube base 410 that mechanically supports one end of the distribution tubes 220a and 220b, and through which the coupling agent passes on its way to the film sheet-transducer surface interface. The transducer surface, described further below, is identified as a cover layer 414. Also shown in FIG. 4B is a rigid PET plastic nosepiece 416 that supports and laterally houses the linear transducer array. The cover layer 414 is flat and is substantially coplanar with the upper edge of the PET plastic lip 404. The cover layer 414 therefore makes gentle contact with the film sheet 216 when it is tautly placed over the PET plastic lip 404.

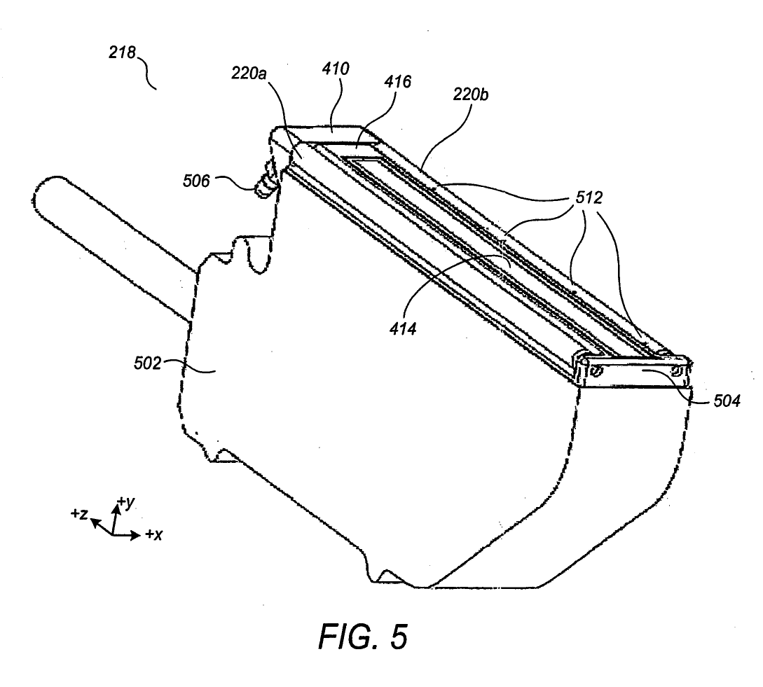

FIG. 5 illustrates a perspective view of the probe assembly 218 according to a preferred embodiment. In this preferred embodiment, there are four (4) 1-mm holes 512 located along the distribution tube 220b, and four corresponding holes (not visible in FIG. 5) along the distribution tube 220a. The inner dimension of the distribution tubes should be relatively wide (e.g., 5 mm) compared to the size of the holes 512 so that a substantially constant pressure is maintained along the distribution tubes. Probe assembly 218 comprises a rigid housing 502 that is manufactured as a laterally separable hollow frame having an opening at nosepiece 416. The transducer array assembly is then placed inside the housing 502, with cover layer 414 protruding through the nosepiece 416. Also shown in FIG. 5 is a support mount 504 for supporting the distal ends of the distribution tubes 220a and 220b, as well as a liquid intake port 506 that couples to tygon tubing for receiving coupling agent from the recycling reservoir.

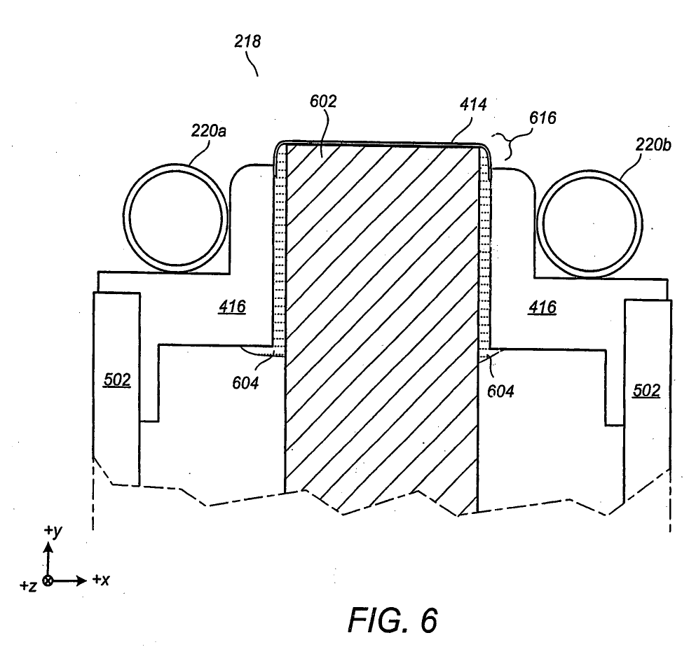

FIG. 6 illustrates an axial cutaway view of the probe assembly 218. Any of a variety of probe materials and construction techniques applicable to linear ultrasound probes may be used to realize the electrical and acoustic properties of a transducer array assembly 602 shown in FIG. 6. Examples include, but are not limited to, techniques described in the following references, each of which is incorporated by reference herein: US20030032884A1; US20030166745A1; U.S. Pat. No. 5,553,035; U.S. Pat. No. 6,014,898; U.S. Pat. No. 6,038,752; U.S. Pat. No. 6,514,618; and U.S. Pat. No. 6,607,491. It is desirable for the probe to be about 15 cm long so as to allow imaging of even large breasts in a single lateral sweep. However, in other preferred embodiments, multiple shorts conventional probes can be placed end-to-end to achieve a similar result. A preferable nominal focus distance is between 1.5 cm and 2.5 cm.

The transducer array assembly 602 is affixed to the nosepiece 416 using general purpose two-part epoxy 604. The nosepiece 416 is rigidly affixed to the housing 502 and forms side ridges that support the distribution tubes 220a and 220b. The top of the cover layer 414 is preferably positioned about 1 mm above an upper rim of the nosepiece 416, as indicated in FIG. 6, and fabricated so as to have an arcuate corner region 616 that facilitates wickable/capillarity-based introduction of couplant between the cover layer 414 and the film sheet 216.

According to a preferred embodiment, the cover layer 414 that covers the transducer assembly 602 comprises a 3-mil sheet of extruded ULTEM® 1000. ULTEM® 1000 is a thermoplastic polyetherimide high heat polymer that, although initially designed for injection molding processing, can also be extruded into film sheets as thin as 3 mils. The 3-mil ULTEM® 1000 sheet is bendable but partially rigid. The cover layer 414 serves multiple purposes including protection of the transducer assembly 602, serving as a matching layer along the acoustic path, and facilitating wicking, wetting, and/or capillary action between itself and the film sheet 216 for optimizing acoustic coupling into the breast. ULTEM® 1000 can be characterized as having high mechanical durability, high heat resistance, a low dissipation factor, and broad chemical resistance. Although 3-mil ULTEM® 1000 is preferred, materials having analogous physical and chemical properties can be substituted.

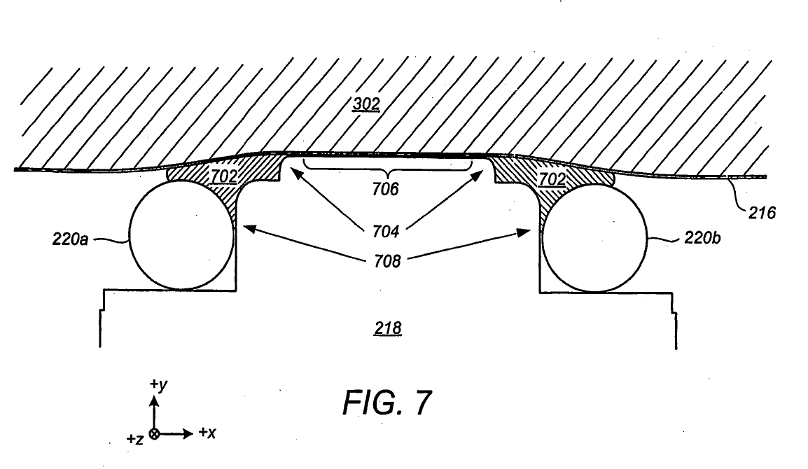

FIG. 7 illustrates a conceptual cutaway axial view of the probe assembly 218 and the film sheet 216 as a breast is being scanned. A dynamic reservoir 702 is formed in the small gap between the film sheet 216, the distribution tube 220b, a corner area 704 of the cover layer 414, and a side surface of 708 of the nosepiece 416. The presence and maintenance of the dynamic reservoir 702 ensures wickable, capillarity-based wetting at an interface 706 between the cover layer 414 and film sheet 216. The dynamic reservoir 702 is dynamic in that there is usually a small amount of coupling agent coming in, and a small amount of coupling agent seeping/weeping out, at any given time. As illustrated in FIG. 7, there is some deformation of the film sheet 216 on either side of the interface 806 due to the physical pressure from the breast 302 above.

In general, the corner area 704 should extend convexly into the dynamic reservoir 702 in a manner that encourages the above wicking/capillary action into the interface 706. The particular convex shape can be circular, having a radius of curvature lying in the range of 0.5 mm-3 mm, or can be of a higher order shape such as a parabola, hyperbola, etc. In alternative preferred embodiments, although believed to be less effective than the convexly-shaped embodiments, there can be a sharp corner or a diagonal ramp leading up to the interface 706. In each case, the interface should be bubble-free, and the film sheet 216 should not “inflate” or rise above the surface of the cover sheet 414 at the interface 706 due to pressure from the coupling agent.

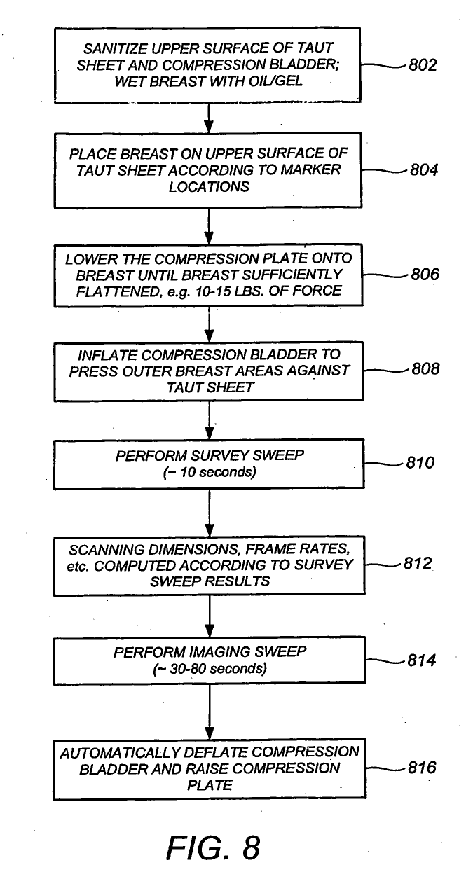

FIG. 8 illustrates step for performing an FFBU scan of a breast according to a preferred embodiment. At step 802 the top surface of the film sheet 216 and the lower surface of the bladder 202 are cleaned and sanitized by using, for example, a sani-wipe. Contact surfaces of the patient's breast and/or the film sheet 216 are coated with a thin layer of oil, gel, or other acoustic coupling agent. Alternatively, to avoid the need for getting the breast wet with such liquid acoustic couplant, an ultrasound couplant sheet can be placed atop the film sheet 216. One kind of ultrasound couplant sheet is the Hydroscan Sterile Couplant Sheet available from Cone Instruments, Inc. of Solon, Ohio.

At step 804, the breast is placed across the top surface of the film sheet 216 according to guide markings printed thereon and/or provided on the translucent compression assembly 112. At step 806, the compression assembly 112 is lowered so that the breast is substantially flattened by the compression plate 204 onto the film sheet 216 using, for example, 10-15 pounds of force. At step 808, the bladder 202 is inflated (to between 0.25-1.0 psi, for example) to press the breast periphery against the film sheet 216. At step 810 the survey sweep described supra is performed, and at step 812 the scanning dimensions, acquisition parameters, etc. as described supra are performed. At step 814 the imaging sweep is performed. At step 816, the bladder 202 is deflated, preferably automatically, and the compression plate is lifted, preferably automatically.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, while the taut mylar sheet is described supra as being fixedly attached to the rigid frame of the scanning chassis, thereby requiring sanitizing wipes between patients, in other preferred embodiments the taut mylar sheet may be disposable such that each patient uses a new taut mylar sheet. Each disposable mylar sheet may be provided in its own lightweight, disposable plastic frame that is inset into grooves provided at the top periphery of the scanning chassis and then removed after the scanning process is complete for each patient. Alternatively, one long sheet of mylar may be provided on a source roller assembly placed on one side of the scanning chassis and received on an uptake roller on the other side. After each patient, the uptake roller may be rotated so as to advance the mylar sheet to a new section for the next patient.

By way of further example, in an alternative preferred embodiment, the compression assembly 112 can be replaced by a second scanning assembly for achieving two-sided scanning of the breast. By way of further example, the scanning assembly 114 can be equipped with a permanent or semi-permanent ultrasound couplant sheet atop the film sheet 216.

By way of even further example, while described supra as using ULTEM® for the cover layer of the probe and Melinex® for the film sheet, the ULTEM® and Melinex® having been found to be well-matched acoustically and to facilitate acoustic coupling between the breast and the first matching layer of the probe, it is to be appreciated that other materials may be substituted. For example, ULTEM® could be used in both the film sheet and the cover layer, or Melinex® could be used in both the film sheet and the cover layer. A variety of different selections and/or combinations of materials can be used for the film sheet and cover layers provided that they are substantially acoustically matched to each other and have the respective properties described supra in this specification.

By way of still further example, although brass distribution tubes are used in the preferred embodiments supra to distribute coupling agent along the transducer surface in distribution manifold arrangement, a variety of different plumbing arrangements achieving the same goal can be provided. Examples include, but are not limited to, “soaker hose” type distribution schemes, nebulizer-type arrangements, misting or gentle-sprinkling arrangements, intermittent sprinkling arrangements (e.g., before the scan but not during the scan). In still another alternative preferred embodiment, the film sheet comprises and/or is treated/coated on the transducer-facing surface to create a hydrophilic surface that further facilitates capillary/wicking action in the acoustic path.

By way of even further example, in another preferred embodiment, the closed chamber formed by the scanning assembly housing and taut film sheet is completely filled with coupling agent. In this preferred embodiment, the chamber itself serves as its own recycling mechanism, the coupling agent never leaving the chamber. Optionally, an external reservoir and accumulator can be provided that replaces any loss of liquid in the filled chamber and that maintains a constant liquid pressure therein. Therefore, reference to the details of the preferred embodiments are not intended to limit their scope, which is limited only by the scope of the claims set forth below.

What is claimed is:

1. A breast scanning apparatus, comprising:
an ultrasound transducer having a scanning surface;
a first compressive member comprising an at least partially conformable membrane in a substantially taut state, said membrane having a first surface for contacting a breast and a second surface opposite said first surface;
a second compressive member, wherein at least one of said first and second compressive members is movable relative to the other to allow placement and compression of the breast between the first and second compression members;
a transducer translation mechanism configured to hold the scanning surface of the ultrasound transducer against said second surface of said membrane while translating the ultrasound transducer thereacross to scan the breast; and
an irrigation system automatically maintaining a continuous supply of coupling agent at an interface between said scanning surface and said second surface of said membrane as said ultrasound transducer is translated thereacross.

2. The breast scanning apparatus of claim 1, said coupling agent comprising a substantially nonviscous liquid, said breast scanning apparatus further comprising a coupling agent recycling system that collects coupling agent departing said interface and returns the coupling agent to said irrigation system for reapplication to said interface.

3. The breast scanning apparatus of claim 2, said first and second compressive members being rotatable around an anterior-posterior axis of a patient for facilitating breast scans at different scan angles including a CC angle, an MLO angle, and an ML angle, wherein said coupling agent recycling system is configured to collect and return said leaving coupling agent to said irrigation system at any of said different scan angles.

4. The breast scanning apparatus of claim 3, further comprising a frame sealably enclosing said ultrasound transducer in cooperation with said membrane for preventing loss of said nonviscous liquid coupling agent.

5. The breast scanning apparatus of claim 4, wherein said nonviscous liquid coupling agent consists primarily of water.

6. The breast scanning apparatus of claim 2, wherein said ultrasound transducer is a linear array transducer.

7. The breast scanning apparatus of claim 6, said irrigation system comprising a distribution tube positioned adjacent to said ultrasound transducer along a length thereof, said distribution tube having openings therealong emitting said coupling agent, wherein a leaking reservoir abutting said interface is established in an elongate gap bounded by said distribution tube, said ultrasound transducer, and said membrane.

8. The breast scanning apparatus of claim 1, wherein said scanning surface comprises a material substantially acoustically matched to said membrane.

9. The breast scanning apparatus of claim 8, wherein said scanning surface comprises a thermoplastic polyetherimide material, and wherein said membrane comprises a biaxially oriented polyester film.

10. The breast scanning apparatus of claim 1, said second compressive member comprising a substantially rigid plate that applies most of a total compression weight to the breast, said second compressive member further comprising and an inflatable bladder that applies a remainder of the total compression weight to the breast in a peripheral area near a skinline of the compressed breast.

11. A method for ultrasonically scanning a breast being compressed by a thin compressive member having a first side and a second side, a first side of said compressive member contacting the breast, comprising:
maintaining a surface of a transducer in contact with the second side of the compressive member while translating the transducer thereacross under motor control to scan the breast; and
automatically irrigating an interface between said transducer surface and the second side of the compressive member in a manner that maintains a continuous supply of coupling agent at said interface.

12. The method of claim 11, said coupling agent comprising a substantially nonviscous liquid that generally departs said interface as said transducer is translated, further comprising automatically recycling said coupling agent and reapplying the recycled coupling agent to said interface.

13. The method of claim 12, further comprising heating said coupling agent to approximately body temperature to facilitate comfort of a patient.

14. The method of claim 12, further comprising filtering said recycled coupling agent prior to said reapplying to said interface.

15. The method of claim 12, said transducer being a linear array transducer, wherein said irrigating comprises maintaining a dynamic reservoir in a gap formed between said transducer, said second surface, and an elongate distribution tube positioned along a length of said transducer near said interface.

16. In a full-field breast ultrasound (FFBU) scanning unit having a first compressive member, the FFBU scanning unit compressing a breast against a first surface of the first compressive member while translating a linear transducer along a second surface thereof opposite the first surface, the linear transducer comprising elements extending in an axial direction and being translated in a lateral direction generally perpendicular to said axial direction, a method for breast scanning, comprising:
performing a full-resolution imaging sweep capturing full-resolution ultrasound frames of the compressed breast at closely-spaced transducer locations corresponding to a nominal lateral image volume resolution;
prior to said full-resolution imaging sweep, performing a survey sweep capturing lower-resolution ultrasound frames at more coarsely-spaced transducer locations;
processing said lower-resolution ultrasound frames to determine a lateral extent of the compressed breast; and
during said full-resolution imaging sweep, skipping over lateral regions of said second surface corresponding to areas outside the lateral extent of the breast, thereby reducing a completion time of said full-resolution imaging sweep.

17. The method of claim 16, further comprising:
processing said lower-resolution ultrasound frames to determine an axial extent of the compressed breast; and
during said full-resolution imaging sweep, deactivating those transducer elements corresponding to axial areas outside the axial extent of the breast, thereby further reducing said completion time.

18. The method of claim 17, said FFBU scanning unit comprising a second compressive member compressing the breast against the first compressive member, said first and second compressive members being separated by a first distance corresponding to a compressed breast thickness, said FFBU scanning unit only processing acoustic interrogation signals for image locations within said first distance from the ultrasound transducer, thereby further reducing said completion time

19. The method of claim 18, wherein said lower-resolution frames are further processed to establish ultrasound acquisition parameters optimizing image quality of said full-resolution frames acquired during said full-resolution imaging sweep.

20. The method of claim 19, wherein said first compressive member comprises a substantially non-stretchable film sheet in a substantially taut state.
